(19) United States
(12) Patent Application Publication
(10) Pub. No.: US 2026/0097168 A1
(43) Pub. Date: Apr. 9, 2026

(54) NEEDLELESS SYRINGE

(71) Applicant: MITAKA HDGS CO., LTD., Mitaka-shi, Tokyo (JP)

(72) Inventor: Manabu HAZAWA, Mitaka-shi (JP)

(73) Assignee: MITAKA HDGS CO., LTD., Mitaka-shi, Tokyo (JP)

(21) Appl. No.: 19/110,317

(22) PCT Filed: Sep. 11, 2023
(86) PCT No.: PCT/JP2023/033074

§ 371 Date: Dec. 29, 2025

(30) Foreign Application Priority Data
Sep. 12, 2022 (JP) 2022-144920

(51) Int. Cl. A61M 5/30; A61M 5/20

(52) U.S. Cl. CPC A61M 5/30; A61M 5/2046

(57) ABSTRACT

A needleless syringe that may administer a fixed amount of liquid in multiple doses in a continuous manner including a plunger and a piston for pushing out the liquid from a liquid container are provided in a cylinder. Gas introduced in a gas supply part may flow into an airtight space by the opening operation of a valve mechanism linked to the pushing of an upper second operating lever, and a piston may move and push a pressing member. When the liquid injection based on a certain stroke is completed, the gas in the airtight space may be released outside a casing by a gas release mechanism. The piston and the pressing member may move back, and the pressing member, which has multiple pressing surfaces with different axial positions, may be rotated by a pressing surface change mechanism to change the pressing surface.

### TECHNICAL FIELD

This invention relates to a needleless syringe that administers a liquid (including the concept of a solution) directly to subcutaneous tissue and the like by spraying it from the tip of a container.

### BACKGROUND ART

A needleless syringe, for example, administers a drug solution to the subcutaneous tissue layer by spraying a drug solution from the spray nozzle at the tip of the nozzle in contact with the skin surface, penetrating the stratum corneum and epidermal cell layer of the skin. Because such a needleless syringe does not have a needle, it does not cause the pain of needle penetration, and it greatly contributes to improving the image of injections. Patent Document 1 discloses a needleless intradermal injection device that comprises a pair of handles that are rotated towards each other to compress a main spring, and a push button that is pressed down to release the compressed state of the main spring. When the compressed state of the main spring is released, the hammer (plunger) moves at high speed due to the spring pressure and the liquid medicine in the container (ampoule) is sprayed out of the nozzle in one go.

### RELATED ART DOCUMENTS

### Patent Documents

[Patent Document 1] Japanese Unexamined Patent Application Publication No. 2015-500131

### DISCLOSURE OF THE INVENTION

### Problem to Be Solved by the Invention

As described above, in the past, the compression energy of the spring was released all at once to administer the entire amount of the liquid medicine from the ampoule at once. In other words, the so-called one-shot method was used. However, depending on the target of the liquid medicine, it may be desirable to administer it in small amounts to multiple locations. If you try to deal with this using the conventional method described above, you will need to prepare multiple ampoules containing small amounts of liquid, and you will also need to repeat the spring compression, perform releasing action, and carry out ampoule replacement each time, which will all be a very troublesome and annoying task.

This invention has been accomplished in light of the above points, and its object is to provide a needleless syringe that can be used to administer a fixed amount of liquid in multiple doses in a continuous manner with simple operation, while having a simple and compact structure, and thereby improving convenience during an actual use.

### Means for Solving the Problem

In order to achieve the above object, the needleless syringe (2) of this invention comprises: a tubular casing (8) with a mounting portion (6) for a liquid container (4) on the tip side thereof; a piston (10) that is movable back and forth in the axial direction of the casing (8); a gas supply portion (14) that is provided behind the piston (10) at the rear end of the casing (8) and supplies gas (12) that moves the piston (10) toward the tip of the casing (8); a valve mechanism (16) that opens and closes the gas supply from the gas supply portion (14); pressing member (18) that is provided in the casing (8) on the tip side of the piston (10) so as to be capable of reciprocating in the axial direction, has a plurality of pressing surfaces (Pf1 to Pf8) whose axial positions differ in stages, and can press a plunger (22) that pushes out the liquid (20) from the liquid container (4) toward the tip side of the casing (8) by a certain stroke(S) in conjunction with the forward movement of the piston (10), and then can move back; a pressing surface changing mechanism (24) that drives the pressing member (18) to change the pressing surface (Pf1 to Pf8) each time the pressing member (18) is returned; a gas release mechanism (26) that releases the gas (12) remaining between the piston (10) and the gas supply part (14) to the outside of the casing (8) every time a pressing operation of a certain stroke(S) is completed.

According to the needleless syringe of the present invention, the gas pressure moves the piston to inject the liquid, and by changing the pressing surface, continuous liquid injection can be performed in stages, so that the work of replacing the ampoule or compressing the spring each time is unnecessary, and the convenience of use can be improved.

In addition, in the above-mentioned needleless syringe (2), the pressing member (18) is provided in the casing (8) in a rotatable manner around an axis parallel to the axial direction, and has a structure in which multiple pressing surfaces (Pf1 to Pf8) are provided in a stepwise manner in the circumferential direction. Meanwhile, pressing surface changing mechanism (24) comprises: a ratchet gear (70) integrally provided with the pressing member (18); and a first operating lever (76) provided in the casing (8) and movable in a direction that intersects the axial direction; a ratchet lever (78) provided on the first operating lever (76) and enabling the ratchet gear (70) to rotate by a predetermined angle in accordance with movement of the first operating lever (76); and a ratchet pawl (80) that stops reverse rotation of the ratchet gear (70). According to this, the pressing surface can be switched in stages with a simple operation of pushing inwardly and releasing the first operating lever.

In addition, the above-mentioned needleless syringe (2) is characterized by the fact that it is provided with a second operating lever (90) that is movable in a direction intersecting the axial direction and is provided in the casing (8), and the valve mechanism (16) is configured such that it is in the open state when the second operating lever (90) is moved and is in the closed state when the second operating lever (90) is moved further. According to this, it is possible to easily open and close the gas supply by simply pushing the second operating lever.

In addition, with the above-mentioned needleless syringe (2), the gas release mechanism (26) is set to the release position by further moving the second operating lever (90) after the valve mechanism (16) has reached the closed state. This allows the used gas to be released at the predetermined timing by a simple operation of inwardly pushing the second operating lever.

In addition, regarding the above-mentioned needleless syringe (2), the first operating lever (76) and the second operating lever (90) are arranged in positions facing each other in the radial direction of the casing (8), and each is arranged parallel to the outer surface of the casing (8), and each can be pushed in the radial direction via an elastic member (88, 92), and the elastic force of the elastic member (88) for the first operating lever (76) is set to be smaller than the elastic force of the elastic member (92) for the second operating lever (90). According to this, a series of actions from changing the pressing surface to releasing gas can be continuously caused by simply squeezing the casing.

Further, the above-mentioned needleless syringe (2) is provided with a first return spring (68) that causes the piston (10) to move back after gas is released by the gas release mechanism (26), and a second return spring (74) that causes the pressing member (18) to move back, while the elastic forces of the first return spring (68) and the second return spring (74) are set so that the piston (10) returns faster than the pressing member (18). According to this, the piston does not apply any load to the pressing member during its return, and the elastic force of the second return spring is prevented from being larger than necessary.

### Effect of the Invention

According to the present invention, a certain amount of liquid can be administered in a continuous manner in a predetermined amount in multiple doses with simple operation, thereby improving convenience during use.

### BRIEF DESCRIPTION OF DRAWINGS

FIG. 1 is an outline cross-sectional view of a needleless syringe of one embodiment of the present invention.

FIG. 2 is a perspective view of the pressing member of the needleless syringe shown in FIG. 1.

FIG. 3 is a front view of the pressing member shown in FIG. 2, viewed from the axial direction.

FIG. 4 is an exploded view of the multiple pressing surfaces of the pressing member shown in FIG. 2.

FIG. 5 is an outline cross-sectional view taken along the X-X line shown in FIG. 1, with some details omitted.

FIG. 6 is an enlarged cross-sectional view of the main parts of the needleless syringe shown in FIG. 1, with the valve mechanism being in an open state.

FIG. 7 is an outline cross-sectional view showing a condition where the plunger is pressed a certain distance and liquid is sprayed, starting from the state shown in FIG. 1.

FIG. 8 is an enlarged cross-sectional view of the main parts of the needleless syringe, showing a state that the valve mechanism is closed and the gas release mechanism is opened, starting from a condition shown in FIG. 6.

### EMBODIMENTS FOR CARRYING OUT THE INVENTION

The following is a detailed description of the embodiment of the present invention, with reference to the attached drawings.

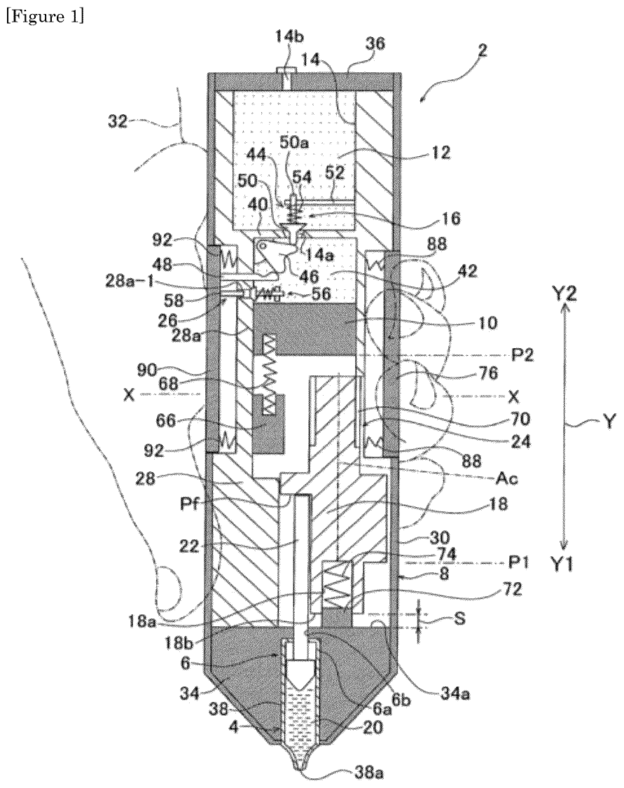

As shown in FIG. 1, the needleless syringe 2 of the present embodiment has: a tubular casing 8 with a mounting portion 6 for a liquid container 4 at the tip end thereof (lower end of the figure); a piston 10 that is movable back and forth in the axial direction (arrow Y direction) of the casing 8; a gas supply portion 14 that is provided at the rear end side (arrow Y2 side) behind the piston 10 in the casing 8 and supplies gas 12 to move the piston 10 toward the front end side (arrow Y1 side) of the casing 8; a valve mechanism 16 that opens and closes (switches on and off) the gas supply from the gas supply portion 14; a pressing member 18 that is provided in the casing 8 so as to be capable of reciprocating in the axial direction at a position closer to the tip end than the piston 10, and has a plurality of pressing surfaces Pf whose axial positions are stepwise different, and that presses a plunger 22, which pushes out the liquid 20 in the liquid container 4, toward the tip side of the casing 8 by a certain stroke in accordance with the forward movement of the piston 10 and then moves back; a pressurizing surface changing mechanism 24 that changes the pressurizing surface Pf by driving the pressing member 18 each time the pressing member 18 reciprocates; and a gas release mechanism 26 that releases the gas 12 accumulating between the piston 10 and the gas supply portion 14 outside the casing 8 each time the pressing operation with a fixed stroke is completed.

The casing 8 has: a cylinder 28 whose inner cross-sectional shape at each axial position is different from one another; and a cover 30 that covers the outer surface of the cylinder 28. The size of the needleless syringe 2 is such that it can be held in one hand (here, the right hand) 32, as shown by the double-dotted line. Although the right hand is shown here, the same applies to use with the left hand. In this embodiment, for example, a cover 30 of the casing 8 can be formed from a synthetic resin material, and the rest of the casing other than the cover can be formed from a metal material, but the present invention is not limited to this, and it is also possible to form all the main parts of the casing from a synthetic resin material.

At the front and rear ends of the casing 8, there are a front block 34 and a rear block 36 with a thinner wall than the front block 34, in such a manner that both ends of the cylinder 28 can be sealed. A mounting portion 6 for the liquid container 4 is formed in the front block 34. The liquid container 4 has a container 38 that contains the liquid 20 for the intended use, and a plunger 22 that pushes the liquid 20 from the container 38 and sprays it out of a nozzle 38a formed at the front end of the container 38. The mounting portion 6 has a fixing portion 6a for fixing the rear end of the container 38 by screwing or press fitting, and an insertion hole 6b for inserting the plunger 22 into the cylinder 28.

The gas supply portion 14 is an airtight space that is partitioned by the front wall 40 and the rear end block 36 serving as the rear wall, and is all on the rear end side of the cylinder 28. In addition, a gas supply port 14a is formed in the front wall 40, and a gas filling port 14b is formed in the rear end block 36. Gas 12 is introduced into the gas supply portion 14 via the gas filling port 14b from an unillustrated gas cylinder. The airtight space 42 between the piston 10 and the front wall 40 is the working space for the gas 12.

The valve mechanism 16 comprises: an opening/closing valve 44 that opens and closes the gas supply port 14a; a pivoting member 46 that is supported in the airtight space 42 in a pivoting manner; and a drive rod 48 that is fixed to the second operating lever 90 described below and makes the pivoting member 46 rotate such that the opening/closing valve 44 opens. The opening and closing valve 44 comprises: a valve body 50 that seals the gas supply port 14a in airtight state, a support plate 52 that is fixed within the gas supply portion 14 and supports the axial portion 50a of the valve body 50 in a sliding manner in the axial direction of the casing 8; and a spring 54 that exerts a force on the valve body 50 in the direction of closing the gas supply port 14a. The space between the valve body 50 and the gas supply port 14a is sealed with a seal member not shown in the Figure.

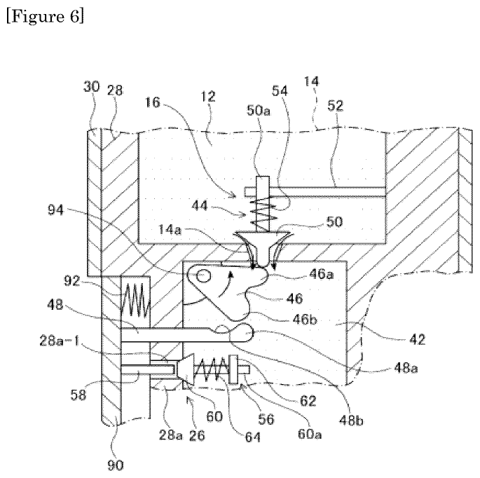

The gas release mechanism 26 comprises: a gas outlet 28a-1 formed in the side wall 28a of the airtight space 42; an opening/closing valve 56 that opens and closes the gas outlet 28a-1 from the inside of the airtight space 42; and a drive rod 58 that is fixed to the second operating lever 90 and pushes the opening/closing valve 56 to open it. As shown in FIG. 6, which is an enlarged view of the relevant parts thereto, the opening and closing valve 56 comprises: a valve body 60 that seals the gas outlet 28a-1 in airtight state; a support plate 62 that is fixed to the inner wall of the airtight space 42 and pivotably supports the axial portion 60a of the valve body 60 in a direction that is perpendicular to the axial direction of the casing 8; and a spring 64 that exerts a force on the valve body 60 in the direction of sealing the gas outlet 28a-1. The space between the valve body 60 and the gas outlet 28a-1 is sealed by a seal member not shown in the Figure.

As shown in FIG. 1, a first return spring 68 is arranged between the piston 10 and the block 66 fixed in the cylinder 28 to return the piston 10 after gas is released by virtue of the gas release mechanism 26.

The pressing member 18 is provided to be rotatable around the axial center Ac parallel to the axial direction within the casing 8, and has a configuration in which multiple pressing surfaces Pf are provided in a stepped (helical staircase-like) manner in the circumferential direction. A spring housing hole 18a is formed at the tip of the pressing member 18, and a ratchet gear 70 is integrally formed at the rear end thereof. A cylindrical spring seat 72 is fixed to the inner surface 34a of the tip block 34, and a part of the spring seat 72 is a state of entering the spring housing hole 18a at the return position P1 of the pressing member 18 shown in FIG. 1. The spring housing hole 18a is provided with a second return spring 74 that returns the pressing member 18 to the return position P1. The distance S between the inner surface 34a of the tip block 34 and the tip surface 18b of the pressing member 18 is a constant stroke when the pressing member 18 reciprocates. This is referred to below as the constant stroke S.

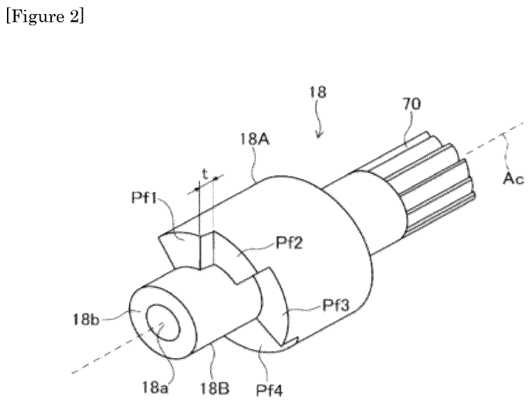

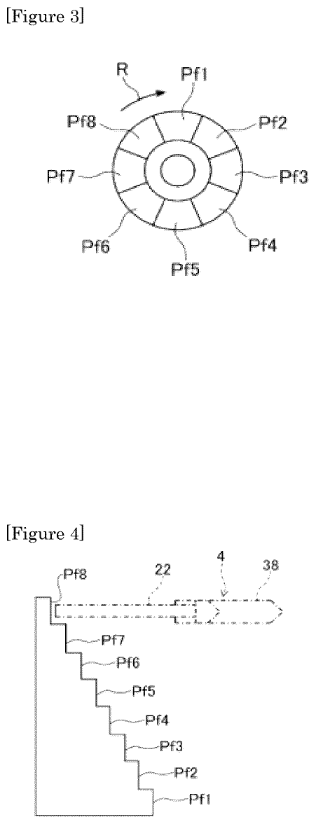

As shown in FIGS. 2 and 3, the pressing member 18 comprises a large diameter portion 18A and a small diameter portion 18B that extends so as to go through the center of the large diameter portion 18A, and the pressing surfaces Pf1 to Pf8 are formed in a stepped spiral staircase shape at different axial positions on the large diameter portion 18A. In other words, each pressing surface Pf1 to Pf8 is formed so that it can be displaced in the circumferential direction while being offset in the axial direction by a dimension t. A ratchet gear 70 is integrally formed at the rear end of the small diameter portion 18B.

FIG. 4 is a planar development of each pressing surface Pf1 to Pf8 of the large diameter portion 18A. When the liquid container 4 is attached, the pressing member 18 is set so that the pressing surface Pf8, which is the farthest from the tip surface 18b in the axial rearward direction (arrow Y2 side), faces the plunger 22. Each time the pressing member 18 is rotated by the pressing surface changing mechanism 24 by a predetermined angle θ (in this case, 45°) in the clockwise direction (arrow R direction) as shown in FIG. 3, the pressing surface Pf is displaced by a dimension t toward the tip of the casing 8, and finally, the plunger 22 is pressed by the pressing surface Pf1, thereby using up all of the liquid 20 contained in the liquid container 4. In this embodiment, the number of pressing surfaces Pf is set to 8, but this number can be changed as appropriate according to the type of liquid 20 and the purpose of use.

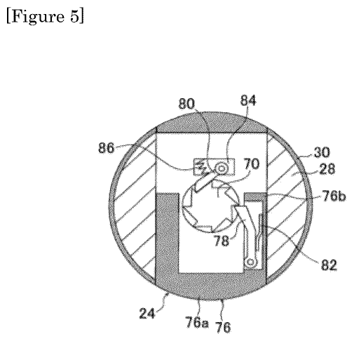

Referring to FIGS. 1 and 5, the structure of the pressing surface changing mechanism 24 will be explained. As shown in FIG. 5, the pressing surface changing mechanism 24 comprises: a ratchet gear 70 integrally provided with the pressing member 18; a first operating lever 76 provided with the casing 8 and movable in a direction intersecting (here, orthogonal to) the axial direction; and a ratchet lever 78 provided on the first operating lever 76, which rotates the ratchet gear 70 by a predetermined angle in conjunction with the pushing movement of the first operating lever 76; and a ratchet pawl 80 that stops the reverse rotation of the ratchet gear 70.

The first operating lever 76 is formed in the shape of a roughly U-shaped cross-portion, and the bottom surface 76a is arranged so that it is nearly flush with the cover 30, which is the outer surface of the casing 8. The ratchet lever 78 is rotatably provided on the inwardly convex portion 76b of the first operating lever 76, and is spring-forced toward the ratchet gear 70 by the leaf spring 82. The ratchet pawl 80 is rotatably provided with a support plate 84 fixed inside the casing 8, and is spring-forced toward the ratchet gear 70 by a spring 86. As shown in FIG. 1, a recess is formed in the part of the casing 8 corresponding to the first operating lever 76, and the first operating lever 76 is returned after pushing inwardly by two coil springs (elastic members) 88 arranged between the first operating lever 76 and the casing 8.

For example, if the middle, ring and little fingers of one hand 32 are placed on the first operating lever 76 and pushed inwards radially towards the casing 8, the pressing member 18 will rotate by 45° together with the ratchet gear 70, and the pressing surface Pf can be switched to the adjacent pressing surface Pf. By repeating the action of pushing inwardly and releasing the first operating lever 76, the pressing surfaces Pf8 to Pf1 can be switched in stages.

As shown in FIG. 1, the needleless syringe 2 has a second operating lever 90 that is provided in the casing 8 and can be moved in a direction that intersects (in this case, is orthogonal to) the axial direction. The second operating lever 90 is a component that operates the valve mechanism 16 and the gas release mechanism 26, and is arranged in a position that is opposite to the first operating lever 76 in the radial direction of the casing 8. Similar to the first operating lever 76, the second operating lever 90 is also arranged so that it is parallel to and flush with the cover 30, which is the outer surface of the casing 8. A recess is formed in the part of the casing 8 corresponding to the second operating lever 90, and the second operating lever 90 is returned after being pushed inwardly by the two coil springs (elastic members) 92 arranged between the second operating lever 90 and the casing 8. The second operating lever 90 can be pushed inwardly with the thumb or the base of the thumb of one hand 32. The elastic force of the coil spring 88 for the first operating lever 76 is set to be smaller than the elastic force of the coil spring 92 for the second operating lever 90.

Referring to FIGS. 6 to 8, the following description will be given to the operation and so on of the valve mechanism 16 and the gas release mechanism 26 by pushing the second operating lever 90 (movement).

As shown in FIG. 6, the oscillating member 46 of the valve mechanism 16 is provided with a protrusion 46a corresponding to the valve main body 50 and a protrusion 46b corresponding to the drive rod 48, and is rotatable about the axis 94.

When the second operating lever 90 is pushed inwardly, the protruding tip 48a of the drive rod 48 comes into contact with the protruding tip 46b, and this causes the oscillating member 46 to rotate in a counterclockwise direction, and the protruding tip 46a presses against the valve body 50. This pressure causes the valve body 50 to move upwards in the figure, and the gas supply port 14a opens, allowing gas 12 to be supplied from the gas supply portion 14 to the airtight space 42. In this state, the gas release mechanism 26 is not yet activated. In other words, the drive rod 58 of the gas release mechanism 26 has not yet come into contact with the valve body 60.

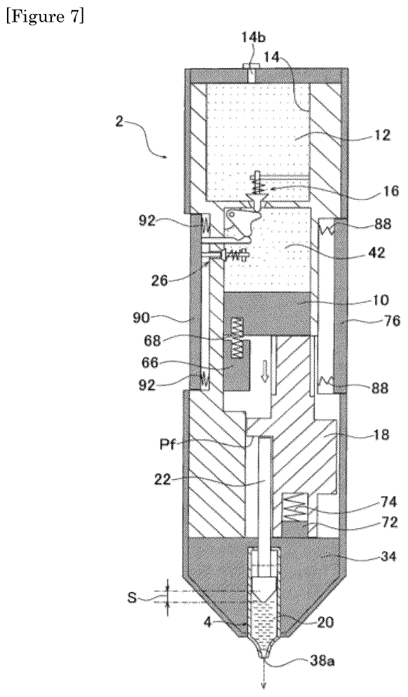

When gas 12 is supplied from the gas supply portion 14 to the airtight space 42, as shown in FIG. 7, the piston 10 is pushed by the pressure of the gas 12 and moves (in the forward direction) to the tip side of the casing 8, and it comes into contact resiliently with the rear end of the pressing member 18 and makes the pressing member 18 move to the tip side. The pressure member 18 moves instantaneously (in the forward direction) until its front surface 18b hits the inner surface 34a of the front block 34, and as a result, the plunger 22 is pushed inwardly at high speed by the pressing surface Pf over a certain stroke S, and a dose of the liquid 20 corresponding to this certain stroke S is sprayed out of the nozzle 38a.

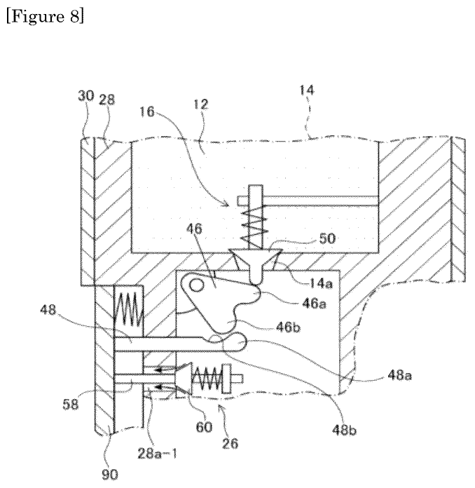

The dose of the liquid 20 is sprayed based on the above-mentioned constant stroke S, as shown in FIG. 6, at the same time as the drive rod 48 of the valve mechanism 16 oscillates the oscillating member 46 and the gas supply port 14a opens. When the second operating lever 90 is pushed inwardly further and the drive rod 48 moves further, as shown in FIG. 8, the protrusion 46b of the oscillating member 46 corresponds to the recess 48b of the drive rod 48, and the oscillating member 46 oscillates in a clockwise direction to close the gas supply port 14a with the valve body 50. The opening and closing of the gas supply port 14a by the valve body 50 is performed continuously within a short period of time. In other words, the valve mechanism 16 is configured such that it is in the open state when the second operating lever 90 is moved, and it is in the closed state when the second operating lever 90 is moved further.

When the pushing movement of the second operating lever 90 is further advanced, at the timing when the liquid 20 spraying operation based on the above-mentioned constant stroke S is completed, the drive rod 58 of the gas release mechanism 26 presses the valve main body 60, the gas outlet 28a-1 opens, and the gas 12 that has been retained in the airtight space 42, i.e., the gas 12 that has moved the piston 10, will be released outside the casing 8. In other words, after the valve mechanism 16 has reached its closed position, the movement of the second operating lever 90 is further advanced to set the gas release mechanism 26 at the release position.

As a result, the pressure in the airtight space 42 decreases, and as shown in FIG. 1, the piston 10 is moved back to the retraction position P2 by the first return spring 68, and the pressing member 18 is moved back to the retraction position P1 by the second return spring 74. The retraction position P1 is the reference position at which the pressing surface Pf of the pressing member 18 is switched. In addition, the spring force of the first return spring 68 is set to be greater than the spring force of the second return spring 74, so that the piston 10 returns before the pressure member 18 returns. The gas 12 is released by the gas release mechanism 26 each time the pressing member 18 completes a certain stroke S of pressure against the plunger 22.

After the release of gas 12 by the gas release mechanism 26 is completed, the push operation of the second operating lever 90 is released and the second operating lever 90 is returned to a position flush with the cover 30 of the casing 8 by the force of the coil spring 92. In this case, the protruding part 48a of the drive rod 48 of the valve mechanism 16 comes into contact with the protruding part 46b of the oscillating member 46, but the oscillating member 46 is prevented from oscillating upwards by an unillustrated projection, and the drive rod 48 slightly elastically deforms to pass through the protruding part 46b. Prior to this, the valve body 60 of the gas release mechanism 26 closes the gas outlet 28a-1 off by virtue of the retraction of the drive rod 58, and the airtight space 42 is made airtight in preparation for the next gas supply. These operations are not performed by the person operating the needleless syringe 2 while being consciously aware of each stage of the operation, but are performed automatically by the operator simply pushing inwardly and releasing the second operating lever 90.

As described above, the elastic force of the coil spring 88 for the first operating lever 76 is set to be smaller than the elastic force of the coil spring 92 for the second operating lever 90. For this reason, as shown in FIG. 1, when the needleless syringe 2 is grasped and gripped in one hand 32, the first operating lever 76 against which the middle finger, ring finger, and little finger are in contact moves first, and the pressing member 18 rotates by a predetermined angle θ due to the operation of the pressing surface changing mechanism 24 accompanying this movement, so that the next pressing surface Pf7 faces the plunger 22. When the first operating lever 76 approaches the cylinder 28 and the pushing force (resistance) increases, the second operating lever 90, which is pressed by the thumb or the base of the thumb, starts to push, and the dose of the liquid 20 is injected based on the above-mentioned constant stroke S.

In other words, from the second pressing surface Pf7 onwards, by simply holding and squeezing the needleless syringe 2 in one hand 32, the injection cycle of switching the pressing surface Pf, supplying the gas 12, injecting a fixed amount of the liquid 20, and releasing the gas 12 can be automatically and continuously performed until the liquid 20 is used up. If the axial position of the pressing surface Pf8 is set to be the same position as the pressing surface Pf7, and the pressing surface Pf8 is set to be such a surface that does not press the plunger 22, namely, such setting position is for setting the liquid container 4 to the needleless syringe 2, then the continuous injections caused by the above-mentioned holding and squeezing operation can be performed at each of the pressing surface Pf7 to Pf1. In this case, for example, the position of the 0th pressing surface Pf8 can be marked in red so that the setting position can be confirmed from the outside of the casing 8, thus making it easier and more reliable to set the liquid container 4 and perform subsequent injection operations.

As in Patent Document 1 and the like., in conventional needleless syringes, the basic structure is to compress a coil spring with a large spring constant and press the plunger with the restoring energy of the coil spring, and for this reason, a large operating force is essential to compress the coil spring. In the needleless syringe 2 of this embodiment, as described above, since the plunger 22 is configured so as to be pressed by the pressure of the gas 12 (including the concepts of compressible gas and liquefied gas), it is sufficient to just only provide an opening/closing structure of the gas 12 and a gas release mechanism 26, and it is possible to simplify and make a structure of the needleless syringe 2 compact, while also reducing the operating force and improving convenience when using it.

In the case of the needleless syringe 2 of this embodiment, since it is possible to perform rapid firing by simply squeezing the casing 8, without the need to prepare multiple containers 4 each of which contains a small amount of liquid 20 and replace them each time of injections and then compress the spring, it is possible to greatly improve the efficiency of use from the perspective of labor and speed.

For example, stem cell supernatant fluid can be used as liquid 20, and the needleless syringe 2 can be used for hair transplantation and subsequent maintenance. In addition to stem cell supernatant fluid, liquid 20 can also be used for vaccines, drugs, cell fluid, blood, papillary hair fluid, gels, suspensions, exosomes and the like.

For example, if a liquid containing stem cell supernatant or exosomes is injected into a skin of a transplanted area of a patient who has undergone a hair transplant surgery, it can be expected to improve the fixation rate of rooting of the transplanted hair. In this embodiment, the needleless syringe 2 makes it possible to inject stem cell supernatant or the like into the skin of the transplanted area on the forehead. According to the present embodiment describing the needleless syringe 2, it is possible to inject a small amount of liquid repeatedly and continuously, so that it is suitable for injecting liquid into the skin of the hair transplanted area over a relatively wide area. In addition, since it is possible to perform injection continuously without using electricity and the like, it is possible for the patient to use this needleless syringe in order to care for the skin area at home or the like, after the hair transplant surgery is performed. Furthermore, the needleless syringe 2 has a compact body and no connected wires or the like, so that it is possible to easily care for the skin area without choosing a location, making it possible to provide a needleless syringe with extremely high convenience.

In addition, in the present embodiment of the needleless syringe 2, the depth of the subcutaneous injection can be changed by changing the diameter (inner diameter) of the nozzle (its tip portion) 38a of the container 38. In other words, if the diameter of the nozzle 38a of the container 38 is increased, the depth to which the liquid 20 is injected under the skin can be kept shallow, while if the diameter of the nozzle 38a of the container 38 is decreased, the depth to which the liquid 20 is injected under the skin can be increased. Therefore, it is possible to inject the liquid to the desired depth by setting the diameter of the nozzle 38a. In addition, for example, by making the tip of the nozzle 38a tapered so that it expands outward, it is possible to adjust the injection range of the liquid 20 (the width and shape of the area to be injected).

The above is an explanation of the embodiment of the present invention, but the present invention is not limited to the above embodiment, and various changes are possible within the scope of the technical ideas described in the claims, the description, and the drawings. For example, in the above embodiment, the gas supply portion 14 is configured to supply the gas 12 from the outside, but it may also be configured to attach a small disposable gas cylinder. It may also be used by connecting the gas supply portion 14 and an external stationary gas cylinder with a gas hose. In addition, although the structure is such that the pressing member 18 is rotated in conjunction with the pushing operation of the first operating lever 76, it may also be a structure in which a large-diameter disk is serrated-fitted to the pressing member 18, and a part of the disk is protruded outside the casing 8, and the disk is rotated by pushing it around with a finger to rotate it to a predetermined angle. Further, the lock on the second operating lever 90 may be automatically released when the first operating lever 76 is fully operated, allowing the second operating lever 90 to be pushed inwardly. In this way, there is no need to change the elastic force of the coil springs 88 and 92, and the above-mentioned operations can be reliably performed with a small operating force. In addition, the materials of the components of the needleless syringe 2 are not limited to the above description.

In the above-mentioned embodiment of the needleless syringe 2, the piston is moved by gas pressure to eject the liquid, but the needleless syringe of the present invention can also be configured to move the piston by an electrical structure, for example, with a motor, to eject the liquid. In this case, the power supply for the electrical structure can be configured to be built into the needleless syringe, such as an electricity charger, or it can be connected to an external power supply. In addition, even in the case of a configuration that supplies gas pressure, in addition to the configuration in which the gas supply portion 14 is built into the needleless syringe as in the above embodiment, it is also possible to configure the system so that gas is supplied from an externally-installed gas supply portion via a tube or other means.

In addition, when using the needleless syringe 2 of the above embodiment, it is also possible to perform EMS (Electrical Muscle Stimulation) to provide electrical stimulation to the muscles beforehand so that the injection can be performed effectively. In addition to the configuration of the needleless syringe of this embodiment, it is also possible to adopt a configuration in which the container 38 and the nozzle 38a at its tip can rotate with respect to the main body part, such as the casing 8 of the needleless syringe 2, and if configured in this way, it is possible to inject the liquid while rotating the container 38 and the nozzle 38a at its tip.

1. A needleless syringe comprising:
a tubular casing having a mounting portion at the tip end thereof that can be used to mount a liquid container which includes: a container and a plunger for pushing the liquid contained in the container out of a nozzle;
a piston that is provided in the casing and can reciprocate in the axial direction of the casing;
a gas supply portion that is provided behind the piston at the rear end of the casing, and supplies gas to move the piston toward the tip end of the casing;
an airtight space provided between the piston and the gas supply portion;
a valve mechanism that opens and closes the gas supply from the gas supply portion to the airtight space;
a pressing member that is provided ahead of the piston in the casing and is capable of reciprocating axially in an axical direction thereof and is driven by the piston driven by the gas pressure in the airtight space, and has a plurality of pressing surfaces whose positions in the axial direction are stepwise different from each other;
a pressing surface changing mechanism that drives the pressing member to change the pressing surface each time the pressing member is driven back;
a gas release mechanism that releases the gas that remains in the airtight space outside the casing each time a pressing operation of a fixed stroke is completed,
wherein the pressing member contacts the plunger of the liquid container mounted in the mounting portion on the pressing surface thereof, and after pressing the plunger towards the tip of the casing for the fixed stroke in conjunction with the piston's forward movement, the plunger is returned.

2. The needleless syringe according to claim 1, wherein
the pressing member is provided rotatable in the casing around an axis parallel to the axial direction, and has a configuration in which the multiple pressing surfaces are provided in a stepwise manner in a circumferential direction,
the pressure surface changing mechanism comprises: a ratchet gear integrally provided with the pressing member; a first operating lever provided within the casing and movable in a direction intersecting the axial direction; a ratchet lever provided with the first operating lever and causing the ratchet gear to rotate by a predetermined angle in accordance with a movement of the first operating lever, and a ratchet pawl for stopping a reverse rotation of the ratchet gear.

3. The needleless syringe according to claim 2, comprising:
a second operating lever provided in the casing, and can be moved in a direction that intersects the axial direction;
wherein the valve mechanism includes: a first opening/closing valve that opens and closes the gas supply to the airtight space; an oscillating member that is supported to be pivotable; and a first drive rod that is fixed to the second operating lever and has a protruding tip and a concave portion;
the oscillating member is provided with a first protrusion corresponding to the first opening/closing valve and a second protrusion corresponding to the first drive rod;
when the second operating lever is moved, the tip protrusion of the first drive rod comes into contact with the second protrusion of the oscillating member, causing the oscillating member to oscillate in the first direction, while the first protrusion of the oscillating member presses on the first opening/closing valve, causing the first opening/closing valve to open;
when the first drive rod moves further as the second operating lever moves further, the second protrusion of the oscillating member corresponds to the concave portion of the first drive rod, and the oscillating member oscillates in the second direction opposite to the first direction, and the first opening/closing valve will be in the closed state.

4. The needleless syringe according to claim 3, wherein
the gas release mechanism comprises: a second opening/closing valve that opens or closes the gas discharge of the airtight space; a second drive rod fixed to the second operating lever that presses so as to open the second opening/closing valve, and
the second operating lever is moved further after the first opening/closing valve is in the closed state, the second drive rod presses the second opening/closing valve, and the second opening/closing valve is then opened.

5. The needleless syringe according to claim 4, wherein
the first operating lever and the second operating lever are arranged in a position facing each other in the radial direction of the casing, and the first operating lever and the second operating lever are arranged parallel to the outer surface of the casing, and each being inwardly pushable in the radial direction via an elastic member;
the elastic force of the elastic member for the first operating lever is set to be smaller than the elastic force of the elastic member for the second operating lever.

6. The needleless syringe according to claim 1, wherein the needleless syringe includes: a first return spring that causes the piston to return after gas is released by the gas release mechanism; and a second return spring that causes the pressing member to return, wherein the elastic forces of the first return spring and the second return spring are set so that the piston returns faster than the pressing member.
